SEQUENCE LISTING (4 sequences)

SEQ ID NO: 1 - protein, 65 aa, Homo sapiens
CTVFLDHENANKILNRPKRYNSGKLEEFVQGNLERECMEEKCSFEEAREVFENTERTTEFWKQYV

SEQ ID NO: 2 - protein, 214 aa, Artificial Sequence
  Synthetic
DIQMTQSPSSLSASVGDRVTITCKASRNIERQLAWYQQKPGQAPELLIYQASRKESGVPDRFSGSRYGTDFTLTISSLQPEDIATYYCQQYSDPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 448 aa, Artificial Sequence
  Synthetic
QVQLVESGGGLVQPGGSLRLSCAASGFTFSYYDIQWVRQAPGKGLEWVSSISPSGQSTYYRREVKGRFTISRDNSKNTLYLQMNSLRAEDTAVYYCARRTGREYGGGWYFDYWGQGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQKEMTKNQVSLTCLVKGFYPSDIAVEWESNGQPENNYKTTPPVLDSDGSFFLYSKLTVDKSRWQEGNVFSCSVMHEALHNRYTQKSLSLSP

SEQ ID NO: 4 - protein, 444 aa, Artificial Sequence
  Synthetic
QVQLVQSGSELKKPGASVKVSCKASGYTFTDNNMDWVRQAPGQGLEWMGDINTRSGGSIYNEEFQDRVIMTVDKSTDTAYMELSSLRSEDTATYHCARRKSYGYYLDEWGEGTLVTVSSASTKGPSVFPLAPCSRSTSESTAALGCLVKDYFPEPVTVSWNSGALTSGVHTFPAVLQSSGLYSLSSVVTVPSSSLGTQTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQYNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQESLSLSP

(19) United States
(12) Patent Application Publication
(10) Pub. No.: US 2021/0284715 A1
(43) Pub. Date: Sep. 16, 2021

(54) COAGULATION FACTOR VIII MIMETIC PROTEIN AND USES THEREOF

(71) Applicant: APPLIED STEMCELL, INC., Milpitas, CA (US)

(72) Inventors: Ling-jie KONG, Union City, CA (US); Ruby Yanru TSAI, San Jose, CA (US); Xiuling CHI, San Jose, CA (US)

(21) Appl. No.: 16/499,281

(22) PCT Filed: Feb. 6, 2018
(86) PCT No.: PCT/US2018/017134

§ 371 Date: Sep. 29, 2019

Related U.S. Application Data
(60) Provisional application No. 62/455,029, filed on Feb. 6, 2017.

(51) Int. Cl. C07K 14/755; C07K 16/36

(52) U.S. Cl. CPC C07K 14/755; C07K 16/36; A61K 38/00; C07K 2317/51; C07K 2317/515; C07K 2319/30

(57) ABSTRACT

The present disclosure provides a coagulation factor VIII(FVII) mimetic protein. The FVIII mimetic protein comprises (1) a coagulating factor IX (FIX/FIXa) binding domain comprising a first heavy chain variable region (VHI) and a first light chain variable region (V_{L1}), wherein the V_{H1} and the V_{L1} are derived from an antibody specifically binding to FIX/FIXa; (2) a coagulation factor X (FX) binding domain comprising a second heavy chain variable region (V_{H2}) and a second light chain variable region (V_{L2}), wherein the V_{H2} and the V_{L2} are derived from an antibody specifically binding to FX; and (3) a membrane binding domain.

### CROSS-REFERENCE TO RELATED APPLICATIONS

This application claims priority to U.S. provisional patent application No. 62/455,029, filed Feb. 6, 2017, the disclosure of which is incorporated herein by reference.

### FIELD OF THE INVENTION

The present invention generally relates to compositions and methods for treating hemophilia.

### BACKGROUND

Hemophilia A is increased incidence of bleeding caused by a genetic deficiency of coagulation factor VIII (FVIII). Produced in liver sinusoidal cells and endothelial cells, FVIII circulates in the bloodstream in an inactive form, bound to von Willebrand factor, until an injury that damages blood vessels occurs. In response to the injury, FVIII is activated and separates from von Willebrand factor to interact with factor IXa and activate factor X in the coagulation cascade. Without binding to von Willebrand factor, activated FVIII is proteolytically degraded and quickly cleared from the blood stream.

Current treatments for hemophilia A are generally limited to administration of FVIII. The half-life of FVIII in blood is about 12 to 16 hours. Hence regular or continuous intravenous administration of recombinant FVIII is often required to treat hemophilia A or prevent bleeding. Therefore, there is a strong need for therapeutic compositions or methods with fewer burdens than using FVIII.

Recently, new approaches for treating hemophilia A have been explored, including using bi-specific antibodies that functionally substitute for FVIII (see, e.g., US20130330345A1 to Tomoyuki Igawa et al.) and gene therapy to introduce a functional FVIII gene to a patient (see, e.g., Nienhuis A W et al., Hum Gene Ther. 2016 27(4):305-8)). The therapeutic effects of these approaches, however, still wait to be confirmed in light of the technical difficulties being faced. For example, the bi-specific antibodies may affect the half-life of FIX, FIXa or FX, the potential of the bi-specific antibodies may be limited due to the inability to interact with the platelet membrane, and the size of FVIII gene (cDNA over 7 kb) has been a major obstacle for gene therapy of hemophilia A. Therefore, there is a continuing need to develop new compositions and methods for treating hemophilia A.

### SUMMARY OF INVENTION

In one aspect, the present disclosure provides a coagulation factor VIII (FVIII) mimetic protein. In one embodiment, the FVIII mimetic protein comprises (1) a coagulating factor IX (FIX/FIXa) binding domain, said FIX/FIXa binding domain comprising a first heavy chain variable region (V_{H1}) and a first light chain variable region (VU), wherein the V_{H1} and the V_{L1} are derived from an antibody specifically binding to FIX/FIXa; and (2) a coagulation factor X (FX) binding domain, said FX binding domain comprising a second heavy chain variable region (V_{H2}) and a second light chain variable region (V_{L2}), wherein the V_{H2} and the V_{L1} are derived from an antibody specifically binding to FX.

In some embodiments, the FIX/FIXa binding domain and/or the FX binding domain of the FVIII mimetic protein has a form of single chain fragment variable domain. In such case, V_{H1} and V_{L1} and/or V_{H2} and V_{L2} are contained in a single polypeptide and are linked by a linker. In one embodiment, the FVIII mimetic protein comprises a single polypeptide comprising the FIX/FIXa binding domain and the FX binding domain, i.e., the FIX/FIXa binding domain and the FX binding domain are linked by a linker.

In some embodiments, the FVIII mimetic protein has a form of Fab fragment, F(ab′)₂ fragment, or Fv fragment.

In one embodiment, the FVIII mimetic protein further comprises antibody constant regions. In one embodiment, the V_{H1} is operably linked to a first heavy chain constant region (C1H), and the V_{L1} is operably linked to a first light chain constant region (C1L). In one embodiment, the V_{H2} is operably linked to a second heavy chain constant region (C2H), and the V_{L2} is operably linked to a second light chain constant region (C2L). In one embodiment, the C1H and C2H are capable of forming a dimer. In one embodiment, the C1H and C2H comprise a hinge region, a CH2 region, and/or a CH3 region of an antibody, respectively.

In some embodiments, the FVIII mimetic protein has a form of bi-specific antibody consisting of four polypeptides. In such case, the FIX/FIXa binding domain comprises a first polypeptide comprising, from N-terminal to C-terminal, a first heavy chain variable domain (V_{H1}) of an FIX/FIXa antibody operably linked to a first heavy chain constant region (C1H) of an antibody, and a second polypeptide comprising, from N-terminal to C-terminal, a first light chain variable domain (V_{L1}) of the FIX/FIXa antibody operably linked to a first I chain constant region (C1H) of an antibody. The FX binding domain comprises a third polypeptide comprising, from N-terminal to C-terminal, a second heavy chain variable domain (V_{H2}) of an FX antibody operably linked to a second heavy chain constant region (C2H) of an antibody, and a fourth polypeptide comprising, from N-terminal to C-terminal, a second light chain variable domain (V_{L2}) of the FX antibody operably linked to a second light chain constant region (C2L) of an antibody; wherein the C1H and C2H are capable of forming a dimer.

In one embodiment, the FVIII mimetic protein of the present disclosure further comprises (3) a membrane binding domain. In one embodiment, the membrane binding domain of the present disclosure binds to platelet membrane. In certain embodiments, the membrane binding domain is a C1, C2 domain, a PH domain, a gamma-carboxyglutamic acid-rich (GLA) domain or membrane binding domain of a platelet membrane glycoprotein. In one embodiment, the membrane binding domain is a C1 or C2 domain derived from FV or FVIII. In one embodiment, the membrane binding domain is derived from GLA domain derived from FII, FVII, FIX, FX, protein C, protein S or protein Z. In one embodiment, the membrane binding domain is derived from an apple3 domain of FXI. In one embodiment, the membrane binding domain is derived from that of AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPlb-IX-V), glycoprotein VI (GPVI), glycoprotein Ia/Ha complex (GPIa/IIa), glycoprotein Ib/Ia complex (GPIIb/IIIa), or GPV/IIIa (GPV/IIa).

In one embodiment, the membrane binding domain binds to lipid membrane through a membrane lipid or through a membrane protein. In certain embodiments, the platelet membrane protein is AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPIb-IX-V), glycoprotein VI (GPVI), glycoprotein Ia/Ia complex (GPIa/IIa), glycoprotein IIb/IIIa complex (GPIIb/IIIa), or GPV/IIIa (GPV/IIa).

In one embodiment, the membrane binding domains are operably linked to the N terminal of the V_{H1} and/or V_{H2}. In one embodiment, the membrane binding domains are operably linked to the C terminal of the C1H and/or C2H. In one embodiment, the membrane binding domains are operably linked to the N terminal of the V_{H1} and/or V_{H2}, and to the C terminal of the C1H and/or C2H.

In one embodiment, the membrane binding domains are operably linked to the N terminal of the V_{L1} and/or V_{L2}. In one embodiment, the membrane binding domains are operably linked to the C terminal of the C1L and/or C2L. In one embodiment, the membrane binding domains are operably linked to the N terminal of the V_{L1} and/or V_{L2}, and to the C terminal of the C1L and/or C2L.

In one embodiment, the V_{L2} and V_{L1} are identical, and/or the C1L and C2L are identical. In one embodiment, the polypeptides in the form of V_{L1}-C1L are identical to that of V_{L2}-C2L.

In some embodiments, the membrane binding domain(s) is operably linked to
1) the N terminal of V_{H2};
2) the C terminal of C2H;
3) both the N terminal of V_{H2} and the C terminal of C2H;
4) the N terminal of V_{H1};
5) both the N terminals of V_{H1} and V_{H2};
6) the N terminal of V_{H1} and the C terminal of C2H;
7) both the N terminals of V_{H1} and V_{H2}, and the C terminal of C2H;
8) the C terminal of V_{H1};
9) the C terminal of C1H and the N terminal of V_{H2};
10) both the C terminals of C1H and C2H;
11) both the C terminals of C1H and C2H, and the N terminal of V_{H2};
12) both the N of V_{H1} and the C terminal of C1H;
13) both the N terminals of V_{H1} and V_{H2}, and the C terminal of C1H;
14) both the C terminals of C1H and C2H, and the N terminal of V_{H1};
15) both the C terminals of C1H and C2H, and both the N terminal of V_{H1} and V_{H2};
16) both the N terminals of V_{L1} and V_{L2};
17) both the N terminals of V_{L1} and V_{L2}, and the N terminal of V_{H2};
18) both the N terminals of V_{L1} and V_{L2}, and the C terminal of C2H;
19) both the N terminals of V_{L1} and V_{L2}, the N terminal of V_{H2}, and the C terminal of C2H;
20) both the N terminals of V_{L1} and V_{L2}, and the N terminal of V_{H1};
21) both the N terminals of V_{L1} and V_{L2}, and both the N terminal of V_{H1} and V_{H2};
22) both the N terminals of V_{L1} and V_{L2}, the N terminal of V_{H1}, and the C terminal of C2H;
23) both the N terminals of V_{L1} and V_{L2}, both the N terminal of V_{H1} and V_{H2}, and the C terminal of C2H;
24) both the N terminals of V_{L1} and V_{L2}, and the C terminal of C1H;
25) both the N terminals of V_{L1} and V_{L2}, the N terminal of V_{H2}, and the C terminal of C1H;
26) both the N terminals of V_{L1} and V_{L2}, and both the C terminal of C1H and C2H;
27) both the N terminals of V_{L1} and V_{L2}, both the C terminal of C1H and C2H, and the N terminal of V_{H2};
28) both the N terminals of V_{L1} and V_{L2}, the N terminal of V_{H1}, and the C terminal of C1H;
29) both the N terminals of V_{L1} and V_{L2}, both the N terminal of V_{H1} and V_{H2}, and the C terminal of C1H;
30) both the N terminals of V_{L1} and V_{L2}, both the C terminal of C1H and C2H, and the N terminal of V_{H1};
31) both the N terminals of V_{L1} and V_{L2}, both the C terminal of C1H and C2H, and both the N terminal of V_{H1} and V_{H2};
32) both the C terminals of C1L and C2L;
33) both the C terminals of C1L and C2L, and the N terminal of V_{H2};
34) both the C terminals of C1L and C2L, and the C terminal of C2H;
35) both the C terminals of C1L and C2L, the N terminal of V_{H1}, and the C terminal of C2H;
36) both the C terminals of C1L and C2L, and the N terminal of V_{H1};
37) both the C terminals of C1L and C2L, and both the N terminals of Vin and V_{H2};
38) both the C terminals of C1L and C2L, the N terminal of V_{H1}, and the C terminal of C2H;
39) both the C terminals of C1L and C2L, both the N terminals of V_{H1} and V_{H2}, and the C terminal of C2H;
40) both the C terminals of C1L and C2L, and the C terminal of C1H;
41) both the C terminals of C1L and C2L, the C terminal of C1H, and the N terminal of V_{H2};
42) both the C terminals of C1L and C2L, and both the C terminals of C1H and C2H;
43) both the C terminals of C1L and C2L, both the C terminals of C1H and C2H, and the N terminal of V_{H2};
44) both the C terminals of C1L and C2L, the N terminal of V_{H1}, and the C terminal of C1H;
45) both the C terminals of C1L and C2L, both the N terminals of V_{H1} and V_{H2}, and the C terminal of C1H;
46) both the C terminals of C1L and C2L, both the C terminals of C1H and C2H, and the N terminal of V_{H1};
47) both the C terminals of C1L and C2L, both the C terminals of C1H and C2H, and both the N terminals of V_{H1} and V_{H2};
48) both the N terminals of V_{L1} and V_{L2}, and both the C terminals of C1L and C2L;
49) both the N terminals of V_{L1} and V_{L2}, both the C terminals of C1L and C2L, and the N terminal of V_{H2};
50) both the N terminals of V_{L1} and V_{L2}, both the C terminals of C1L and C2L, and the C terminal of C2H;
51) both the N terminals of V_{L1} and V_{L2}, both the C terminals of C1L and C2L, the N terminal of V_{H2}, and the C terminal of C2H;
52) both the N terminals of V_{L1} and V_{L2}, both the C terminals of C1L and C2L, and the N terminal of V_{H2};
53) both the N terminals of V_{L1} and V_{L2}, both the C terminals of C1L and C2L, and both the N terminals of V_{H1} and V_{H2};
54) both the N terminals of V_{L1} and V_{L2}, both the C terminals of C1L and C2L, the N terminal of V_{H1}, and the C terminal of C2H;
55) both the N terminals of V_{L1} and V_{L2}, both the C terminals of C1L and C2L, both of the N terminal of V_{H1} and V_{H2}, and the C terminal of C2H;
56) both the N terminals of V_{L1} and V_{L2}, both the C terminals of C1L and C2L, and the C terminal of C1H;
57) both the N terminals of V_{L1} and V_{L2}, both the C terminals of C1L and C2L, the C terminal of C1H, and the N terminal of V_{H2};
58) both the N terminals of V_{L1} and V_{L2}, both the C terminals of C1L and C2L, and both the C terminal of C1H and C2H;
59) both the N terminals of V_{L1} and V_{L2}, both the C terminals of C1L and C2L, both the C terminal of C1H and C2H, and the N terminal of V_{H2};
60) both the N terminals of V_{L1} and V_{L2}, both the C terminals of C1L and C2L, the N terminal of V_{H1}, and the C terminal of C1H;
61) both the N terminals of V_{L1} and V_{L2}, both the C terminals of C1L and C2L, both the N terminal of V_{H1} and V_{H2}, and the C terminal of C1H;
62) both the N terminals of V_{L1} and V_{L2}, both the C terminals of C1L and C2L, both the C terminal of C1H and C2H; or
63) both the N terminals of V_{L1} and V_{L2}, both the C terminals of C1L and C2L, both the N terminals of V_{H1} and V_{H2}, and both the C terminal of C1H and C2H.

In another aspect, the present disclosure provides a nucleic acid encoding the FVIII mimetic protein as disclosed herein.

In another aspect, the present disclosure provides a vector comprising the nucleic acid as disclosed herein.

In yet another aspect, the present disclosure provides a cell comprising the nucleic acid as disclosed herein.

In another aspect, the present disclosure provides a method for producing the FVIII mimetic protein. In one embodiment, the method comprises culturing the cell as disclosed herein.

In another aspect, the present disclosure provides a pharmaceutical composition comprising the FVIII mimetic protein as disclosed herein and a pharmaceutically acceptable carrier.

In another aspect, the present disclosure provides a kit comprising the FVIII mimetic protein as disclosed herein.

In another aspect, the present disclosure provides a method for treating or reducing the incidence of bleeding, a disease accompanying bleeding, or a disease caused by bleeding in a subject. In one embodiment, the method comprises introducing to a cell of the subject a vector comprising a nucleic acid encoding a coagulation factor VIII (FVIII) mimetic protein. In one embodiment, said FVIII mimetic protein comprising: (1) a coagulating factor IX (FIX/FIXa) binding domain comprising a first heavy chain variable region (V_{H1}) and a first light chain variable region (V_{L1}), wherein the V_{H1} and the V_{L1} are derived from an antibody specifically binding to FIX/FIXa; and (2) a coagulation factor X (FX) binding domain, said FX binding domain comprising a second heavy chain variable region (V_{H2}) and a second light chain variable region (Vu), wherein the V_{H2} and the V_{L2} are derived from an antibody specifically binding to FX.

In certain embodiments, the bleeding, a disease accompanying bleeding, or a disease caused by bleeding is hemophilia A, acquired hemophilia or von Willebrand disease.

In certain embodiments, the cell of the present disclosure is endothelial cell, liver cell, platelet, PBMC or hematopoietic stem cell.

In one embodiment, the subject is a human.

In one embodiment, the vector is an episomal expression vector. In one embodiment, the vector is a donor vector for gene knock-in.

In one embodiment, the vector is introduced to the cell via a virus. In certain embodiments, the virus is an adeno-associated virus, a retrovirus or a lentivirus.

In one embodiment, the method of the present disclosure further comprises introducing to the cell a site-specific nuclease. In certain embodiments, the site-specific nuclease is selected from a CRISPR nuclease, a TALEN, a DNA-guided nuclease and a Zinc Finger nuclease.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the schematic illustration of FV111.

FIG. 2 shows an exemplary schematic illustration of the FVIII mimetic protein and the gene encoding thereof. MBD: membrane binding domain.

FIG. 3 represents the polypeptides of the FVIII mimetic protein with or without the membrane binding domain operably linked to the polypeptides that form the FIX/FIXa binding domain or the FX binding domain; polypeptides 1-3 and 10 represent the first polypeptide of the FVIII mimetic protein that comprises V_{H1} (dark bending rod), with or without the membrane binding domain (circle) operably linked to either or both terminals; polypeptides 4-6 and 11 represent the third polypeptide of the FVIII mimetic protein that cpmprises V_{H2} (light grey bending rod), with or without the membrane binding domain (empty circle) operably linked to either or both terminals; polypeptides 7-9 and 12 represent the second or the fourth polypeptide of the FVIII mimetic protein that comprises V_{L1} and/or V_{L2} (lined short rod), with or without the membrane binding domain operably linked to either or both terminals.

FIGS. 4A and 4B show the various structural combinations of the FIX/FIXa binding domain, the FX binding domain and the membrane binding domains.

FIG. 5 shows the amino acid sequences of factor IX GLA domain and the polypeptides of the FVIII mimetic protein.

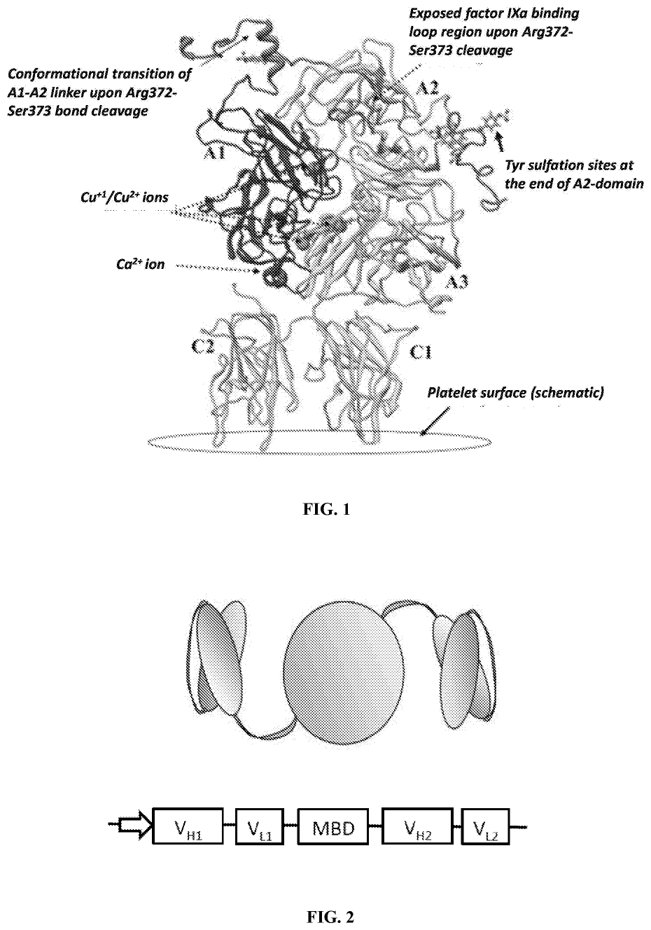

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

The term “antibody” as used herein includes any immunoglobulin, monoclonal antibody, polyclonal antibody, multi-specific antibody, or bispecific (bivalent) antibody that binds to a specific antigen. A native intact antibody comprises two heavy chains and two light chains. Each heavy chain consists of a variable region and a first, second, and third constant region, while each light chain consists of a variable region and a constant region. Mammalian heavy chains are classified as α, δ, ε, γ, and μ, and mammalian light chains are classified as λ or κ. The antibody has a “Y” shape, with the stem of the Y consisting of the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J Mol Biol 273(4):927 (1997); Chothia, C. et al., J Mol Biol 186(3):651-63 (1985); Chothia, C. and Lesk, A. M., J Mol Biol, 196:901 (1987); Chothia, C. et al., Nature 342 (6252):877-83 (1989); Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (al heavy chain), or IgA2 (α2 heavy chain).

The term “antigen-binding domain” as used herein refers to a protein portion that can specifically bind to a particular antigen. An antigen-binding domain may be a portion of an antibody comprising one or more CDRs, or any other antibody fragment that binds to an antigen but does not comprise an intact native antibody structure. Examples of antigen-binding domain include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multispecific antibody, a camelized single domain antibody, a nanobody, a domain antibody, and a bivalent domain antibody. An antigen-binding domain is capable of binding to the same antigen to which the parent antibody binds.

The term “bispecific” as used herein means that, there are two antigen-binding domains, each of which is capable of specifically binding to a different antigen or a different epitope on the same antigen.

Bispecific antigen-binding polypeptides may be produced by chemically crosslinking Fab's or scFv, such as using ortho-phenylenedi-maleimide (o-PDM) as the crosslinking reaction group between the Fab's from different antibodies (Keler T et al. Cancer Research 1997, 57: 4008-4014), or chemically crosslinking an Fab′-thionitrobenzoic acid (TNB) derivative and an antibody fragment such as Fab′-thiol (SH) (Brennan Metal. Science 1985, 229: 81-83).

A heterodimers of the heavy chains derived from different antigen-binding domains can also be generated by a leucine zipper derived from Fos and Jun, via adding a Fos leucine zipper to one of the antigen-binding domain, such as Fab's, scFvs, or Fvs, etc and adding a Jun leucine zipper to the other (Kostelny S A et al. J. of Immunology, 1992, 148: 1547-53).

The bispecific antigen-binding polypeptides includes, but not limited to, a sc(Fv)₂, a IgG-scFv, a BiTE, a DVD-Ig, a TriFabs, a Fab-Fab, a Fab-Fv, a MAb-Fv, a IgG-Fv, a ScFab-Fc-scFv2, a ScFab-Fc-scFv, an appended IgG, a CrossMab, and a diabody.

“Single-chain Fv” or “scFv” refers to an engineered antigen-binding domain consisting of an antibody light chain variable region and an antibody heavy chain variable region connected to one another directly or via a peptide linker sequence (Huston J S et al. (1988) Proc NatL Acad Sci USA, 85:5879).

“Fab” with regard to an antibody refers to that portion of the antibody consisting of a single light chain (both variable and constant regions) associating to the variable region and first constant region of a single heavy chain by a disulphide bond. In certain embodiments, the constant regions of both the light chain and heavy chain are replaced with TCR constant regions.

“Fab” refers to a Fab fragment that includes a portion of the hinge region. “F(ab′)₂” refers to a dimer of Fab′.

“Fc” with regard to an antibody refers to that portion of the antibody consisting of the second (CH2) and third (CH3) constant regions of a first heavy chain bound to the second and third constant regions of a second heavy chain via disulphide bonding. The Fc portion of the antibody is responsible for various effector functions such as ADCC, and CDC, but does not function in antigen binding.

“Fv” with regard to an antibody refers to the smallest fragment of the antibody to bear the complete antigen binding site. An Fv fragment consists of the variable domain of a single light chain bound to the variable domain of a single heavy chain. A number of Fv designs have been provided, including dsFvs, in which the association between the two domains is enhanced by an introduced disulphide bond; and scFvs can be formed using a peptide linker to bind the two domains together as a single polypeptide. Fvs constructs containing a variable domain of a heavy or light immunoglobulin chain associated to the variable and constant domain of the corresponding immunoglobulin heavy or light chain have also been produced. Fvs have also been multimerised to form diabodies and triabodies (Maynard et al., Annu Rev Biomed Eng 2 339-376 (2000)).

“TriFabs” refers to a trivalent, bispecific fusion protein composed of three units with Fab-functionalities. TriFabs harbor two regular Fabs fused to an asymmetric Fab-like moiety.

“Fab-Fab” refers to a fusion protein formed by fusing the Fd chain of a first Fab arm to the N-terminus of the Fd chain of a second Fab arm.

“Fab-Fv” refers to a fusion protein formed by fusing a HCVR to the C-terminus of a Fd chain and a LCVR to the C-terminus of a light chain. A “Fab-dsFv” molecule can be formed by introducing an interdomain disulphide bond between the HCVR domain and the LCVR domain.

“MAb-Fv” or “IgG-Fv” refers to a fusion protein formed by fusion of HCVR domain to the C-terminus of one Fc chain and the LCVR domain either expressed separately or fused to the C-terminus of the other resulted in a bispecific, trivalent IgG-Fv (mAb-Fv) fusion protein, with the Fv stabilized by an interdomain disulphide bond.

“ScFab-Fc-scFv2” and “ScFab-Fc-scFv” refer to a fusion protein formed by fusion of a single-chain Fab with Fc and disulfide-stabilized Fv domains.

“Appended IgG” refers to a fusion protein with a Fab arm fused to an IgG to form the format of bispecific (Fab)₂-Fc. It can form a “IgG-Fab” or a “Fab-IgG”, with a Fab fused to the C-terminus or N-terminus of an IgG molecule with or without a connector. In certain embodiments, the appended IgG can be further modified to a format of IgG-Fab4 (see, Brinkman et al., 2017, Supra).

“DVD-Ig” refers to a dual-variable-domain antibody that is formed by fusion of an additional HCVR domain and LCVR domain of a second specificity to an IgG heavy chain and light chain. “CODV-Ig” refers to a related format where the two HCVR and two LCVR domains are linked in a way that allows crossover pairing of the variable HCVR-LCVR domains, which are arranged either (from N- to C-terminus) in the order HCVRA-HCVRB and LCVRB-LCVRA, or in the order HCVRB-HCVRA and LCVRA-LCVRB.

A “CrossMab” refers to a technology of pairing of unmodified light chain with the corresponding unmodified heavy chain and pairing of the modified light chain with the corresponding modified heavy chain, thus resulting an antibody with reduced mispairing in the light chain.

A “BiTE” is a bispecific T-cell engager molecule, comprising a first scFv with a first antigen specificity in the LCVR-HCVR orientation linked to a second scFv with a second specificity in the HCVR-LCVR orientation.

“Hinge region” in terms of an antibody includes the portion of a heavy chain molecule that joins the CH1 domain to the CH2 domain. This hinge region comprises approximately 25 amino acid residues and is flexible, thus allowing the two N-terminus antigen binding regions to move independently.

“CH2 domain” as used herein refers to includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat, E., et al., U.S. Department of Health and Human Services, (1983)).

The CH3 domain extends from the CH2 domain to the C-terminus of the IgG molecule and comprises approximately 108 amino acids. Certain immunoglobulin classes, e.g., IgM, further include a CH4 region.

The term “operably link” or “operably linked” refers to a juxtaposition, with or without a spacer or linker, of two or more biological sequences of interest in such a way that they are in a relationship permitting them to function in an intended manner. When used with respect to polypeptides, it is intended to mean that the polypeptide sequences are linked in such a way that permits the linked product to have the intended biological function. For example, an antibody variable region may be operably linked to a constant region so as to provide for a stable product with antigen-binding activity. The term may also be used with respect to polynucleotides. For one instance, when a polynucleotide encoding a polypeptide is operably linked to a regulatory sequence (e.g., promoter, enhancer, silencer sequence, etc.), it is intended to mean that the polynucleotide sequences are linked in such a way that permits regulated expression of the polypeptide from the polynucleotide.

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, and artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

A cell, as used herein, can be prokaryotic or eukaryotic. A prokaryotic cell includes, for example, bacteria. A eukaryotic cell includes, for example, a fungus, a plant cell, and an animal cell. The types of an animal cell (e.g., a mammalian cell or a human cell) includes, for example, a cell from circulatory/immune system or organ (e.g., a B cell, a T cell (cytotoxic T cell, natural killer T cell, regulatory T cell, T helper cell), a natural killer cell, a granulocyte (e.g., basophil granulocyte, an eosinophil granulocyte, a neutrophil granulocyte and a hypersegmented neutrophil), a monocyte or macrophage, a red blood cell (e.g., reticulocyte), a mast cell, a thrombocyte or megakaryocyte, and a dendritic cell); a cell from an endocrine system or organ (e.g., a thyroid cell (e.g., thyroid epithelial cell, parafollicular cell), a parathyroid cell (e.g., parathyroid chief cell, oxyphil cell), an adrenal cell (e.g., chromaffin cell), and a pineal cell (e.g., pinealocyte)); a cell from a nervous system or organ (e.g., a glioblast (e.g., astrocyte and oligodendrocyte), a microglia, a magnocellular neurosecretory cell, a stellate cell, a boettcher cell, and a pituitary cell (e.g., gonadotrope, corticotrope, thyrotrope, somatotrope, and lactotroph)); a cell from a respiratory system or organ (e.g., a pneumocyte (a type I pneumocyte and a type II pneumocyte), a clara cell, a goblet cell, an alveolar macrophage); a cell from circular system or organ (e.g., myocardiocyte and pericyte); a cell from digestive system or organ (e.g., a gastric chief cell, a parietal cell, a goblet cell, a paneth cell, a G cell, a D cell, an ECL cell, an I cell, a K cell, an S cell, an enteroendocrine cell, an enterochromaffin cell, an APUD cell, a liver cell (e.g., a hepatocyte and Kupffer cell)); a cell from integumentary system or organ (e.g., a bone cell (e.g., an osteoblast, an osteocyte, and an osteoclast), a teeth cell (e.g., a cementoblast, and an ameloblast), a cartilage cell (e.g., a chondroblast and a chondrocyte), a skin/hair cell (e.g., a trichocyte, a keratinocyte, and a melanocyte (Nevus cell)), a muscle cell (e.g., myocyte), an adipocyte, a fibroblast, and a tendon cell), a cell from urinary system or organ (e.g., a podocyte, a juxtaglomerular cell, an intraglomerular mesangial cell, an extraglomerular mesangial cell, a kidney proximal tubule brush border cell, and a macula densa cell), and a cell from reproductive system or organ (e.g., a spermatozoon, a Sertoli cell, a leydig cell, an ovum, an oocyte). A cell can be normal, healthy cell; or a diseased or unhealthy cell (e.g., a cancer cell). A cell further includes a mammalian zygote or a stem cell which include an embryonic stem cell, a fetal stem cell, an induced pluripotent stem cell, and an adult stem cell. A stem cell is a cell that is capable of undergoing cycles of cell division while maintaining an undifferentiated state and differentiating into specialized cell types. A stem cell can be an omnipotent stem cell, a pluripotent stem cell, a multipotent stem cell, an oligopotent stem cell and a unipotent stem cell, any of which may be induced from a somatic cell. A stem cell may also include a cancer stem cell. A mammalian cell can be a rodent cell, e.g., a mouse, rat, hamster cell. A mammalian cell can be a lagomorpha cell, e.g., a rabbit cell. A mammalian cell can also be a primate cell, e.g., a human cell. In certain examples, the cells are those used for mass bioproduction, e.g., CHO cells.

FVIII Mimetic Protein

In one aspect, the present disclosure provides a FVIII mimetic protein that comprises

a coagulating factor IX (FIX/FIXa) binding domain, said FIX/FIXa binding domain comprising a first heavy chain variable region (VH1) and a first light chain variable region (VL1), wherein the VH1 and the VL1 are derived from an antibody specifically binding to FIX/FIXa;

a coagulation factor X (FX) binding domain, said FX binding domain comprising a second heavy chain variable region (VH2) and a second light chain variable region (VL2), wherein the VH2 and the VL2 are derived from an antibody specifically binding to FX; and a membrane binding domain. In one embodiment, the membrane binding domain(s) is linked to any location of the FIX/FIXa binding domain or the FX binding domain that does not affect the binding activity and the bispecificity of the FVIII mimetic protein to FIX/FIXa and FX.

In one embodiment, said FIX/FIXa binding domain comprises a first scFv, and said FX binding domain comprises a second scFv, wherein the first scFv operably links to the second scFv. In one embodiment, the first and second scFv are linked directly. In one embodiment, the first and second scFv are linked via a linker or a polypeptide linker, such as human serum albumin (HSA). In one embodiment, the first and second scFvs are linked between the C terminal of the light chain variable region, and the membrane binding domain(s) is operably linked to

1) the N terminal of V_{H2};

2) the N terminal of V_{H1};

3) both the N terminals of V_{H1} and V_{H2}.

In one embodiment, the first and second scFvs are linked between the C terminal of the heavy chain variable region and the membrane binding domain(s) is operably linked to

1) the N terminal of V_{L1};

2) the N terminal of V_{L1};

3) both the N terminals of V_{L1} and V_{L2}.

In one embodiment, the first and second scFvs are linked between the C terminal of the first light chain variable region and the N terminal of the second light chain variable region, and the membrane binding domain(s) is operably linked to

1) the C terminal of V_{L2};

2) the N terminal of V_{H1};

3) both the N terminal of VI and the C terminal of V_{L2}.

In some embodiments, the FVIII mimetic protein has a form of bi-specific antibody consisting of four polypeptides. In such case, the FIX/FIXa binding domain comprises a first polypeptide comprising, from N-terminal to C-terminal, a first heavy chain variable domain (V_{H1}) of an FIX/FIXa antibody operably linked to a first heavy chain constant region (C1H) of an antibody, and a second polypeptide comprising, from N-terminal to C-terminal, a first light chain variable domain (V_{L1}) of the FIX/FIXa antibody operably linked to a first I chain constant region (C1H) of an antibody. The FX binding domain comprises a third polypeptide comprising, from N-terminal to C-terminal, a second heavy chain variable domain (V_{H2}) of an FX antibody operably linked to a second heavy chain constant region (C2H) of an antibody, and a fourth polypeptide comprising, from N-terminal to C-terminal, a second light chain variable domain (V_{L2}) of the FX antibody operably linked to a second light chain constant region (C2L) of an antibody; wherein the C1H and C2H are capable of forming a dimer.

The FIX/FIXa binding domain is associated with the FX binding domain directly or via a linker or a polypeptide linker.

In one embodiment, the membrane binding domain is operably linked to the FIX/FIXa binding domain and/or the FX binding domain directly or via a linker or a polypeptide linker.

The linkage between the FIX/FIXa binding domain and/or the FX binding domain and the membrane binding domains can be various, for example, the membrane binding domains are operably linked to:
1) the N terminal of V_{H2};
2) the C terminal of C2H;
3) both the N terminal of V_{H2} and the C terminal of C2H;
4) the N terminal of V_{H1};
5) both the N terminals of V_{H1} and V_{H2};
6) the N terminal of V_{H1} and the C terminal of C2H;
7) both the N terminals of V_{H1} and V_{H2}, and the C terminal of C2H;
8) the C terminal of V_{H1};
9) the C terminal of C1H and the N terminal of V_{H2};
10) both the C terminals of C1H and C2H;
11) both the C terminals of C1H and C2H, and the N terminal of V_{H2};
12) both the N of V_{H1} and the C terminal of C1H;
13) both the N terminals of V_{H1} and V_{H2}, and the C terminal of C1H;
14) both the C terminals of C1H and C2H, and the N terminal of V_{H1};
15) both the C terminals of C1H and C2H, and both the N terminal of V_{H1} and V_{H2};
16) both the N terminals of V_{L1} and V_{L2};
17) both the N terminals of V_{L1} and V_{L2}, and the N terminal of V_{H2};
18) both the N terminals of V_{L1} and V_{L2}, and the C terminal of C2H;
19) both the N terminals of V_{L1} and V_{L2}, the N terminal of V_{H2}, and the C terminal of C2H;
20) both the N terminals of V_{L1} and V_{L2}, and the N terminal of V_{H1};
21) both the N terminals of V_{L1} and V_{L2}, and both the N terminal of V_{H1} and V_{H2};
22) both the N terminals of V_{L1} and V_{L2}, the N terminal of V_{H1}, and the C terminal of C2H;
23) both the N terminals of V_{L1} and V_{L2}, both the N terminal of V_{H1} and V_{H2}, and the C terminal of C2H;
24) both the N terminals of V_{L1} and V_{L2}, and the C terminal of C1H;
25) both the N terminals of V_{L1} and V_{L2}, the N terminal of V_{H2}, and the C terminal of C1H;
26) both the N terminals of V_{L1} and V_{L2}, and both the C terminal of C1H and C2H;
27) both the N terminals of V_{L1} and V_{L2}, both the C terminal of C1H and C2H, and the N terminal of V_{H2};
28) both the N terminals of V_{L1} and V_{L2}, the N terminal of V_{H1}, and the C terminal of C1H;
29) both the N terminals of V_{L1} and V_{L2}, both the N terminal of V_{H1} and V_{H2}, and the C terminal of C1H;
30) both the N terminals of V_{L1} and V_{L2}, both the C terminal of C1H and C2H, and the N terminal of V_{H1};
31) both the N terminals of V_{L1} and V_{L2}, both the C terminal of C1H and C2H, and both the N terminal of V_{H1} and V_{H2};
32) both the C terminals of C1L and C2L;
33) both the C terminals of C1L and C2L, and the N terminal of V_{H2};
34) both the C terminals of C1L and C2L, and the C terminal of C2H;
35) both the C terminals of C1L and C2L, the N terminal of V_{H1}, and the C terminal of C2H;
36) both the C terminals of C1L and C2L, and the N terminal of V_{H1};
37) both the C terminals of C1L and C2L, and both the N terminals of V_{H1} and V_{H2};
38) both the C terminals of C1L and C2L, the N terminal of V_{H1}, and the C terminal of C2H;
39) both the C terminals of C1L and C2L, both the N terminals of V_{H1} and V_{H2}, and the C terminal of C2H;
40) both the C terminals of C1L and C2L, and the C terminal of C1H;
41) both the C terminals of C1L and C2L, the C terminal of C1H, and the N terminal of V_{H2};
42) both the C terminals of C1L and C2L, and both the C terminals of C1H and C2H;
43) both the C terminals of C1L and C2L, both the C terminals of C1H and C2H, and the N terminal of V_{H2};
44) both the C terminals of C1L and C2L, the N terminal of V_{H1}, and the C terminal of C1H;
45) both the C terminals of C1L and C2L, both the N terminals of V_{H1} and V_{H2}, and the C terminal of C1H;
46) both the C terminals of C1L and C2L, both the C terminals of C1H and C2H, and the N terminal of V_{H1};
47) both the C terminals of C1L and C2L, both the C terminals of C1H and C2H, and both the N terminals of V_{H1} and V_{H2};
48) both the N terminals of V_{L1} and V_{L2}, and both the C terminals of C1L and C2L;
49) both the N terminals of V_{L1} and V_{L2}, both the C terminals of C1L and C2L, and the N terminal of V_{H2};
50) both the N terminals of V_{L1} and V_{L2}, both the C terminals of C1L and C2L, and the C terminal of C2H;
51) both the N terminals of V_{L1} and V_{L2}, both the C terminals of C1L and C2L, the N terminal of V_{H2}, and the C terminal of C2H;
52) both the N terminals of V_{L1} and V_{L2}, both the C terminals of C1L and C2L, and the N terminal of V_{H1};
53) both the N terminals of V_{L1} and V_{L2}, both the C terminals of C1L and C2L, and both the N terminals of V_{H1} and V_{H2};
54) both the N terminals of V_{L1} and V_{L2}, both the C terminals of C1L and C2L, the N terminal of V_{H1}, and the C terminal of C2H;
55) both the N terminals of V_{L1} and V_{L2}, both the C terminals of C1L and C2L, both of the N terminal of V_{H1} and V_{H2}, and the C terminal of C2H;
56) both the N terminals of V_{L1} and V_{L2}, both the C terminals of C1L and C2L, and the C terminal of C1H;
57) both the N terminals of V_{L1} and V_{L2}, both the C terminals of C1L and C2L, the C terminal of C1H, and the N terminal of V_{H2};
58) both the N terminals of V_{L1} and V_{L2}, both the C terminals of C1L and C2L, and both the C terminal of C1H and C2H;
59) both the N terminals of VU and Via, both the C terminals of C1L and C2L, both the C terminal of C1H and C2H, and the N terminal of V_{H2};
60) both the N terminals of V_{L1} and V_{L2}, both the C terminals of C1L and C2L, the N terminal of V_{H1}, and the C terminal of C1H;
61) both the N terminals of V_{L1} and V_{L2}, both the C terminals of C1L and C2L, both the N terminal of V_{H1} and V_{H2}, and the C terminal of C1H;
62) both the N terminals of V_{L1} and V_{L2}, both the C terminals of C1L and C2L, both the C terminal of C1H and C2H; or
63) both the N terminals of V_{L1} and V_{L2}, both the C terminals of C1L and C2L, both the N terminals of V_{H1} and V_{H2}, and both the C terminal of C1H and C2H.

In one embodiment, the FVIII mimetic protein comprise a second and a fourth polypeptides having the sequence of SEQ ID NO: 2, a first polypeptide having the sequence of SEQ ID NO: 3, and a third polypeptide having the sequence of SEQ ID NO: 4. In one embodiment, the membrane binding domain is a gamma-carboxyglutamic acid-rich (GLA) domain, which is a protein domain that contains post-translational modifications of many glutamate residues by vitamin K-dependent carboxylation to form γ-carboxyglutamate (GLA). Proteins with this domain are known informally as GLA proteins. The GLA residues are responsible for the high-affinity binding of calcium ions. The GLA domain is contained in many human proteins, such as Thrombin, Factor VII, Factor IX, Factor X, Protein C (PROC), Protein S (PROS1), Protein Z (PROZ), osteocalcin (BGLAP), matrix GLA protein (MGP), GAS6, transthyretin (TTR), Inter-alpha-trypsin inhibitor heavy chain H2 (ITIH2), periostin, proline rich GLA1 (PRRG1), proline rich GLA2 (PRRG2), proline rich GLA3 (PRRG3), proline rich GLA4 (PRRG4).

In one embodiment, the GLA domain is derived from Factor IX. The sequence of human gamma-carboxyglutamic acid-rich domain can be obtained from public database such as NCBI (GI No. 157830594 or 157830595). A Factor IX GLA can be obtained via known methods in the art, such as recombinant expression or chemical synthesis (see M Jacobs et al., Membrane binding properties of the factor IX gamma-carboxyglutamic acid-rich domain prepared by chemical synthesis, Journal of Biological Chemistry, 1994, 269 (41):25494-501). In one embodiment, the GLA domain has a sequence as set forth in SEQ ID NO: 1.

In certain embodiments, the FIX/FIXa binding domain and FX binding domain of the FVIII mimetic protein as provided herein is based on the format of a “whole” antibody, such as whole IgG or IgG-like molecules, or small and small recombinant formats, such as tandem single chain variable fragment molecules (taFvs), diabodies (Dbs), single chain diabodies (scDbs) and various other derivatives of these (cf. bispecific antibody formats as described by Byrne H. et al. (2013) Trends Biotech, 31 (11): 621-632.

In certain embodiments, the FIX/FIXa binding domain and FX binding domain of the FVIII mimetic protein as provided herein is based on a bispecific format selected from Triomabs; hybrid hybridoma (quadroma); Multispecific anticalin platform (Pieris); Diabodies; Single chain diabodies; Tandem single chain Fv fragments; TandAbs, Trispecific Abs (Affimed); Darts (dual affinity retargeting; Macrogenics); Bispecific Xmabs (Xencor); Bispecific T cell engagers (Bites; Amgen; 55 kDa); Triplebodies; Tribody (Fab-scFv) Fusion Protein (CreativeBiolabs) multifunctional recombinant antibody derivates; Duobody platform (Genmab); Dock and lock platform; Knob into hole (KIH) platform; Humanized bispecific IgG antibody (REGN1979) (Regeneron); Mab₂ bispecific antibodies (F-Star); DVD-Ig (dual variable domain immunoglobulin) (Abbvie); kappa-lambda bodies; TBTI (tetravalent bispecific tandem Ig); and CrossMab.

In certain embodiments, the FIX/FIXa binding domain and FX binding domain of the FVIII mimetic protein as provided herein is based on a bispecific format selected from bispecific IgG-like antibodies (BsIgG) comprising CrossMab; DAF (two-in-one); DAF (four-in-one); DutaMab; DT-IgG; Knobs-in-holes common LC; Knobs-in-holes assembly; Charge pair; Fab-arm exchange, SEEDbody; Triomab; LUZ-Y; Fcab; kappa-lamda-body; and Orthogonal Fab. For detailed description of the bispecific antibody formats please see Spiess C., Zhai Q. and Carter P. J. (2015) Molecular Immunology 67: 95-106, which is incorporated herein by reference to its entirety.

In certain embodiments, the FIX/FIXa binding domain and FX binding domain of the FVIII mimetic protein as provided herein is based on a bispecific format selected from IgG-appended antibodies with an additional antigen-binding moiety comprising DVD-IgG; IgG(H)-scFv; scFv-(H)IgG; IgG(L)-scFv; scFV-(L)IgG; IgG(L,H)-Fv; IgG(H)-V; V(H)-IgG; IgG(L)-V; V(L)-IgG; KIH IgG-scFab; 2scFv-IgG; IgG-2scFv; scFv4-Ig; scFv4-Ig; Zybody; and DVI-IgG (four-in-one) (see Id.).

In certain embodiments, the FIX/FIXa binding domain and FX binding domain of the FVIII mimetic protein as provided herein is based on a format selected from bispecific antibody fragments comprising Nanobody; Nanobody-HAS; BiTE; Diabody; DART; TandAb; scDiabody; sc-Diabody-CH3; Diabody-CH3; Triple Body; Miniantibody; Minibody; TriBi minibody; scFv-CH3 KIH; Fab-scFv; scFv-CH-CL-scFv; F(ab′)2; F(ab′)2-scFv2; scFv-KIH; Fab-scFv-Fc; Tetravalent HCAb; scDiabody-Fc; Diabody-Fc; Tandem scFv-Fc; and Intrabody (see Id).

In certain embodiments, the FIX/FIXa binding domain and FX binding domain of the FVIII mimetic protein as provided herein is based on a bispecific format such as Dock and Lock; ImmTAC; HSAbody; scDiabody-HAS; and Tandem scFv-Toxin (see Id).

In certain embodiments, the FIX/FIXa binding domain and FX binding domain of the FVIII mimetic protein as provided herein is based on a format selected from bispecific antibody conjugates comprising IgG-IgG; Cov-X-Body; and scFv1-PEG-scFv2 (see Id).

Production of FVIII Mimetic Protein

The FIX/FIXa binding domain and FX binding domain of the FVIII mimetic protein can be derived from different parent antibodies. A parent antibody can be any type of antibody, including for example, a fully human antibody, a humanized antibody, or an animal antibody (e.g. mouse, rat, rabbit, sheep, cow, dog, etc.). The parent antibody can be a monoclonal antibody or a polyclonal antibody.

In one embodiment, the parent antibody is a monoclonal antibody. A monoclonal antibody can be produced by various methods known in the art, for example, hybridoma technology, recombinant method, phage display, or any combination thereof.

Hybridoma technology involves fusion of antibody-expressing B cells with an immortal B cell line to produce hybridomas, which are further screened for desirable characteristics such as high production level of antibody production, good growth of hybridoma cells, and strong binding or good biological activity of the antibody (see, for example, Harlow et al. (1988) Antibodies: A Laboratory Manual, (Cold Spring Harbor Laboratory Press, 2nd ed.).

Recombinant method is another way to produce a parent antibody. Briefly, cells such as lymphocytes secreting antibodies of interest are obtained are identified and single cells are isolated, followed by reverse transcriptase PCR to produce heavy- and light-chain variable region cDNAs. These cDNA sequences of the variable regions can be used to construct the encoding sequence of the polypeptide complex provided herein, and then expressed in a suitable host cell (for reviews, please see, for example, U.S. Pat. No. 5,627,052; PCT Publication No. WO 92/02551; and Babcock et al. (1996) Proc. Natl. Acad. Sci. USA 93:7843-7848).

Antibody libraries are still an alternative for obtaining a parent antibody. Briefly, one can screen an antibody library to identify an antibody having the desired binding specificity. Methods for such screening of recombinant antibody libraries are well known in the art and include methods described in, for example, U.S. Pat. No. 5,223,409; PCT Publication Nos. WO 92/18619; WO 91/17271; WO 92/20791; WO 92/15679; WO 93/01288; WO 92/01047; WO 92/09690; and WO 97/29131; Fuchs et al. (1991) Bio/Technology 9:1370-1372; Hay et al. (1992) Hum. Antibod. Hybridomas 3:81-85; Huse et al. (1989) Science 246:1275-1281; McCafferty et al. (1990) Nature 348:552-554; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natd. Acad. Sci. USA 89:3576-3580; Garrad et al. (1991) Bio/Technology 9:1373-1377; Hoogenboom et al. (1991) Nucl. Acid Res. 19:4133-4137; and Barbas et al. (1991) Proc. Natl. Acad. Sci. USA 88:7978-7982; and US Publication No. 20030186374.

Another illustrative method to obtain a parent antibody is phage display (see, e.g., Brinkman et al. (1995) J. Immunol. Methods 182:41-50; Ames et al. (1995) J. Immunol. Methods 184:177-186; Kettleborough et al. (1994) Eur. J. Immunol. 24:952-958; Persic et al. (1997) Gene 187 9-18; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743; and 5,969,108). Polynucleotide sequences encoding antibody domains are introduced to phage particles to generate a library of phage particles displaying a variety of functional antibody domains. Fd and M13 are filamentous phage commonly used, and the functional antibody domains displayed on the phage particles can be, for example, Fab, Fv or disulfide stabilized Fv antibody domains, which is recombinantly fused to a phage protein encoded by gene III or gene VIII. The phage library can be screened using an antigen of interest, for example, which is optionally labeled or bound or captured to a solid substrate (e.g. a bead). For a selected phage, its polynucleotide sequences encoding the antibody variable domains are obtained and used in the construction of the polypeptide complex provided herein. Likewise, a library of yeast can be generated displaying antibody variable domains by tethering the antibody domains to the yeast cell wall (see, for example, U.S. Pat. No. 6,699,658), and then screened with a bound antigen to obtain a parent antibody useful for construction of the polypeptide complex provided herein.

Furthermore, a parent antibody can also be produced by injecting an antigen of interest to a transgenic non-human animal comprising some, or all, of the human immunoglobulin locus, for example, OmniRat, OmniMouse (see, for example, Osborn M. et al, Journal of Immunology, 2013, 190: 1481-90; Ma B. et al, Journal of Immunological Methods 400-401 (2013) 78-86; Geurts A. et al, Science, 2009, 325:433; U.S. Pat. No. 8,907,157; EP patent 2152880B1; EP patent 2336329B1), HuMab mice (see, for details, Lonberg, N. et al. Nature 368(6474): 856 859 (1994)), Xeno-Mouse (Mendez et al. Nat Genet., 1997, 15:146-156), TransChromo Mouse (Ishida et al. Cloning Stem Cells, 2002, 4:91-102) and VelocImmune Mouse (Murphy et al. Proc Natl Acad Sci USA, 2014, 111:5153-5158), Kymouse (Lee et al. Nat Biotechnol, 2014, 32:356-363), and transgenic rabbit (Flisikowska et al. PLoS One, 2011, 6:e21045).

The parent antibodies described herein can be further modified, for example, to graft the CDR sequences to a different framework or scaffold, to substitute one or more amino acid residues in one or more framework regions, to replace one or more residues in one or more CDR regions for affinity maturation, and so on. These can be accomplished by a person skilled in the art using conventional techniques.

The parent antibody can also be a therapeutic antibody known in the art, for example those approved by FDA for therapeutic or diagnostic use, or those under clinical trial for treating a condition, or those in research and development. Polynucleotide sequences and protein sequences for the variable regions of known antibodies can be obtained from public databases.

The polypeptides of the FVIII mimetic protein can be produced by expressing the gene encoding the corresponding polypeptides alone. Alternatively, the polypeptides can be generated by splitting the parent antibodies protein structure via known methods in the art, such as incubation in a reduced condition to break the interchain disulfide bond.

In one embodiment, the membrane binding domains are operably linked to the N terminal of the V_{H1} and/or V_{H2}. In one embodiment, the membrane binding domains are operably linked to the C terminal of the C1H and/or C2H. In one embodiment, the membrane binding domains are operably linked to the N terminal of the V_{H1} and/or V_{H2}, and to the C terminal of the C1H and/or C2H.

In one embodiment, the membrane binding domains are operably linked to the N terminal of the V_{L1} and/or V_{L2}. In one embodiment, the membrane binding domains are operably linked to the C terminal of the C1L and/or C2L. In one embodiment, the membrane binding domains are operably linked to the N terminal of the V_{L2} and/or V_{L2}, and to the C terminal of the C1L and/or C2L.

In one embodiment, the V_{L1} and V_{L2} are identical, and/or the C1L and C2L are identical. In one embodiment, the polypeptides in the form of V_{L1}-C1L are identical to that of V_{L2}-C2L.

In one embodiment, the membrane binding domains are operably linked to the N terminal and/or C terminal of the first polypeptide. In one embodiment, the membrane binding domains are operably linked to the N terminal and/or C terminal of the second polypeptide. In one embodiment, the membrane binding domains are operably linked to the N terminal and/or C terminal of the third polypeptide. In one embodiment, the membrane binding domains are operably linked to the N terminal and/or C terminal of the fourth polypeptide. In one embodiment, the membrane binding domains are operably linked to the N terminal and/or C terminal of the first, second, third and/or fourth polypeptide.

Protein Purification

In certain embodiments, the FVIII mimetic protein of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70° %, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying a FVIII mimetic protein of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

In some embodiments, complete FVIII mimetic proteins are fractionated utilizing agents (i.e., protein A) that bind the Fc portion of the FVIII mimetic protein. Alternatively, antigens may be used to simultaneously purify and select appropriate FVIII mimetic protein. Such methods often utilize the selection agent bound to a support, such as a column, filter or bead. The FVIII mimetic protein is bound to a support, contaminants removed (e.g., washed away), and the FVIII mimetic protein released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE. It will therefore be appreciated that under differing electrophoresis conditions, the apparent molecular weights of purified or partially purified expression products may vary.

Compositions for Treating Hemophilia A

In one aspect, the present disclosure provides compositions for treating hemophilia A. In one aspect, the composition comprises a coagulation factor VIII (FVIII) mimetic protein that functionally substitute for FVIII. In one embodiment, the FVIII mimetic protein comprises (1) a coagulating factor IX (FIX/FIXa) binding domain, said FIX/FIXa binding domain comprising a first heavy chain variable region (V_{H1}) and a first light chain variable region (V_{L1}), wherein the V_{H1} and the V_{L1} are derived from an antibody specifically binding to FIX/FIXa; and (2) a coagulation factor X (FX) binding domain, said FX binding domain comprising a second heavy chain variable region (V_{H2}) and a second light chain variable region (V_{L2}), wherein the V_{H2} and the V_{L2} are derived from an antibody specifically binding to FX.

As used herein, “coagulation factor VIII” or “FVIII” refers to a blood-clotting protein that has a GenBank Reference ID NP_000123 (preproprotein) or NP_063916. FVIII is an essential component in the coagulation cascade. It is a cofactor for Factor IXa that, in the presence of Ca²⁺ and phospholipids forms a complex that converts Factor X to the activated form Xa. FVIII circulates in the bloodstream in an inactive form, bound to von Willebrand factor. In the event of an injury that damages blood vessels, FVIII is activated and separated from von Villebrand factor. The active FVIII or FVIIIa interacts with FIX to trigger a chain reaction that form a blood clot.

“Factor IX”, or “FIX”, also known as Christmas factor, refers to a serine protease of the coagulation system and has a GenBank Ref. No. NP_000124 or NP_001300842. FIX is produced as an inactive precursor, which is processed to remove the signal peptide, glycosylated and cleaved to produce a two-chain form where the chains are linked by a disulfide bond. The two-chain form is active and also called FIXa.

“Factor X”, or “FX”, also known as Stuart-Prower factor, is a serine endopeptidase of the coagulation cascade and has a GenBank Ref. No. NP_000495, NP-001299603 or NP_001299604.

The term “specific binding” or “specifically binds” as used herein refers to a non-random binding reaction between two molecules, such as for example between an antibody and an antigen. In certain embodiments, the antibodies or antigen-binding fragments provided herein specifically bind human FIX, FIXa or FX with a binding affinity (K_{D}) of ≤10⁻⁶ M (e.g., ≤5×10⁻⁷ M, ≤2×10⁻⁷ M, ≤10⁻⁷ M, ≤5×10⁻⁸ M, ≤2×10⁻⁸ M, ≤10⁻⁸ M, ≤5×10⁻⁹ M, ≤2×10⁻⁹ M, ≤10⁻⁹ M, 10⁻¹⁰ M). K_{D} as used herein refers to the ratio of the dissociation rate to the association rate (k_{off}/kₒₙ), may be determined using surface plasmon resonance methods for example using instrument such as Biacore.

Methods to produce antibodies and variable regions thereof specifically binding to an antigen are known in the art (see, e.g., EA Greenfield, Antibodies A Laboratory Manual, 2^{nd} Ed., Cold Spring Harbor laboratory Press, 2013). Some antibodies specifically binding to FIX/FIXa or FX are known in the art (see, e.g., US20130330345A1 to Tomoyuki Igawa et al.).

In certain embodiments, the FVIII mimetic protein of the present disclosure also comprises (3) a membrane binding domain. As used herein, a “membrane binding domain” refers to a domain that targets a protein to lipid membrane by interacting with lipid membrane or membrane proteins.

In one embodiment, the membrane binding domain of the present disclosure binds to platelet membrane. In some embodiments, the membrane binding domain binds to a membrane through a membrane lipid or through a membrane protein. In certain embodiments, the membrane binding domain is a C1, C2 domain, a PH domain, a gamma-carboxyglutamic acid-rich (GLA) domain or membrane binding domain of a platelet membrane glycoprotein. In one embodiment, the membrane binding domain is a C1 or C2 domain derived from FV or FVIII. In one embodiment, the membrane binding domain is derived from a GLA domain of FII, FVII, FIX, FX, protein C, protein S or protein Z. In one embodiment, the membrane binding domain is derived from an apple3 platelet binding domain of FXI. In one embodiment, the membrane binding domain is derived from that of AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPIb-IX-V), glycoprotein VI (GPVI), glycoprotein Ia/IIa complex (GPIa/IIa), glycoprotein IIb/IIa complex (GPIIb/IHa), or GPV/IIIa (GPV/IIa).

In one embodiment, the membrane binding domain binds to lipid membrane through a platelet membrane protein. In certain embodiments, the platelet membrane protein is AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPIb-IX-V), glycoprotein VI (GPVI), glycoprotein Ia/IIa complex (GPIa/IIa), glycoprotein IIb/IIIa complex (GPIIb/IIIa), or GPV/IIIa (GPV/IIa).

In certain embodiments, the FVIII mimetic protein of the present disclosure also comprises a linker that connects the domains disclosed herein. The linker or polypeptide linker described herein refers to a peptide sequence designed to connect (e.g., join, link) two protein sequences, wherein the linker peptide sequence is typically not disposed between the two protein sequences in nature. In the context of the present invention, the phrase “linked” or “joined” or “connected” generally refers to a functional linkage between two contiguous or adjacent amino acid sequences to produce a polypeptide that generally does not exist in nature. In certain embodiments, linkage may be used to refer to a covalent linkage. Generally, linked proteins are contiguous or adjacent to one another and retain their respective operability and function when joined. Peptides comprising the mimetic protein disclosed herein are linked by means of an interposed peptide linker comprising one or more amino acids. Such linkers may provide desirable flexibility to permit the desired expression, activity and/or conformational positioning of the mimetic protein. A typical amino acid linker is generally designed to be flexible or to interpose a structure, such as an alpha-helix, between the two protein moieties. The linker peptide sequence can be of any appropriate length to connect one or more proteins of interest and is preferably designed to be sufficiently flexible so as to allow the proper folding and/or function and/or activity of one or both of the peptides it connects. Means by which to make fusion and/or chimeric polypeptides are well-known in the art (see, e.g., Sambrook et al., Molecular Cloning: A Laboratory Manual, Cold Springs Harbor Laboratory, 1992, New York).

In another aspect, the present disclosure provides a nucleic acid encoding the FVIII mimetic protein as disclosed herein.

In another aspect, the present disclosure provides a vector comprising the nucleic acid as disclosed herein.

In yet another aspect, the present disclosure provides a cell comprising the nucleic acid as disclosed herein.

In another aspect, the present disclosure provides a method for producing the FVIII mimetic protein. In one embodiment, the method comprises culturing the cell as disclosed herein.

In another aspect, the present disclosure provides a pharmaceutical composition comprising the FVIII mimetic protein as disclosed herein and a pharmaceutically acceptable carrier.

In another aspect, the present disclosure provides a kit comprising the FVIII mimetic protein as disclosed herein.

In another aspect, the present disclosure provides a method for treating or reducing the incidence of bleeding, a disease accompanying bleeding, or a disease caused by bleeding in a subject.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. In certain embodiments, the bleeding, a disease accompanying bleeding, or a disease caused by bleeding is hemophilia A, acquired hemophilia or von Willebrand disease.

In one embodiment, the method comprises introducing to a cell of the subject a vector comprising a nucleic acid encoding a coagulation factor VIII (FVIII) mimetic protein as disclosed herein.

In one embodiment, the vector is an episomal expression vector. In one embodiment, the vector is a donor vector for gene knock-in.

The term “introduce” in the context of inserting a nucleic acid sequence into a cell, means “transfection”, or “transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon. The vector of the present disclosure may be introduced into a cell using any method known in the art. Various techniques for transforming animal cells may be employed, including, for example: microinjection, retrovirus mediated gene transfer, electroporation, transfection, or the like (see, e.g., Keown et al., Methods in Enzymology 1990, 185:527-537). In one embodiment, the vector is introduced to the cell via a virus.

In one embodiment, the method of the present disclosure further comprises introducing to the cell a site-specific nuclease. As used herein, a “nuclease” is an enzyme capable of cleaving the phosphodiester bonds between the nucleotide subunits of nucleic acids. A “nuclease domain” is an independently folded protein domain having nuclease activity. A “site-specific nuclease” refers to a nuclease whose functioning depends on a specific nucleotide sequence. Typically, a site-specific nuclease recognizes and binds to a specific nucleotide sequence and cuts a phosphodiester bond within or in the vicinity of the nucleotide sequence. In certain embodiments, the double-strand break is generated by site-specific cleavage using a site-specific nuclease. Examples of site-specific nucleases include, without limitation, zinc finger nucleases (ZFNs), transcriptional activator-like effector nucleases (TALENs) and CRISPR (clustered regularly interspaced short palindromic repeats)-associated (Cas) nucleases.

In one embodiment, the method of the present disclosure further comprises introducing to the cell a gRNA complement to a targeting DNA sequence. As used herein, a “CRISPR-Cas guide RNA” or “guide RNA” or “gRNA” refers to an RNA that directs sequence-specific binding of a CRISPR complex to the target sequence. Typically, a guide RNA comprises (i) a guide sequence that has sufficient complementarity with a target polynucleotide sequence to hybridize with the target sequence and (ii) a trans-activating cr (tracr) mate sequence. A guide RNA may further comprises a tracr RNA fused at the 3′ end, resulting a single chimeric guide RNA. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

As used herein, a “target DNA sequence” refers to a sequence recognized by the site-specific nuclease domain. In some embodiments, the target DNA sequence is a sequence within a genome of a cell. Exemplary target sequences include those that are unique in the target genome. In some embodiments, a target DNA sequence is located in the nucleus or cytoplasm of a cell. In some embodiments, the target sequence may be within an organelle of a eukaryotic cell, for example, mitochondrion or chloroplast.

In certain embodiments, the nucleic acid encoding the FVIII mimetic protein is incorporated to the genome through the non-homologous end joining (NHEJ) pathway or the homology-directed repair (HDR) (see Moore J K, Haber J E, 1996. “Cell cycle and genetic requirements of two pathways of nonhomologous end-joining repair of double-strand breaks in Saccharomyces cerevisiae”. 16 (5): 2164-73.).

### EXAMPLE

The following example is included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

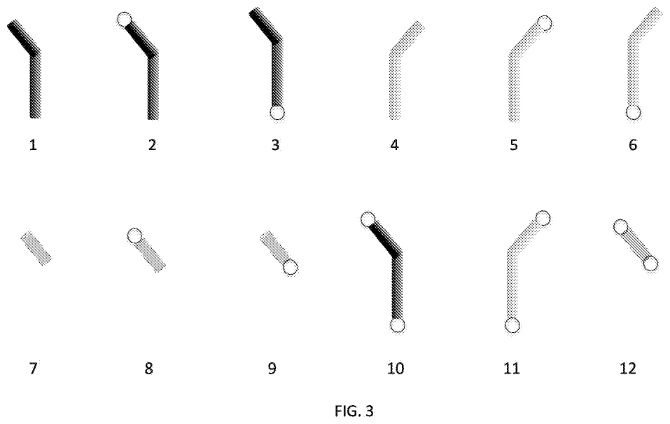

This example illustrates the generation of an FVIII mimetic protein having FXa generation-promoting activity. Twelve animal cell expression vectors containing the nucleic acid sequences of a common shared light chain (SEQ ID NO: 2) and two different heavy chains (anti-human FIXa antibody H chain (SEQ ID NO: 3) and anti-human FX antibody H chain (SEQ ID NO: 4), respectively), with the nucleic acid sequence encoding the GLA domain of FIX (SEQ ID NO: 1) operably linked to various locations are constructed, as illustrated in FIG. 3.

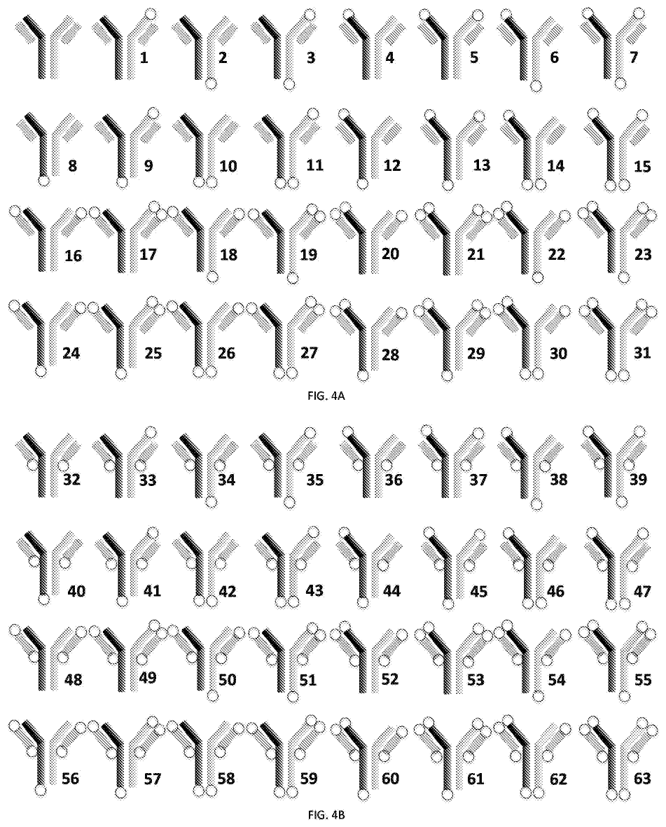

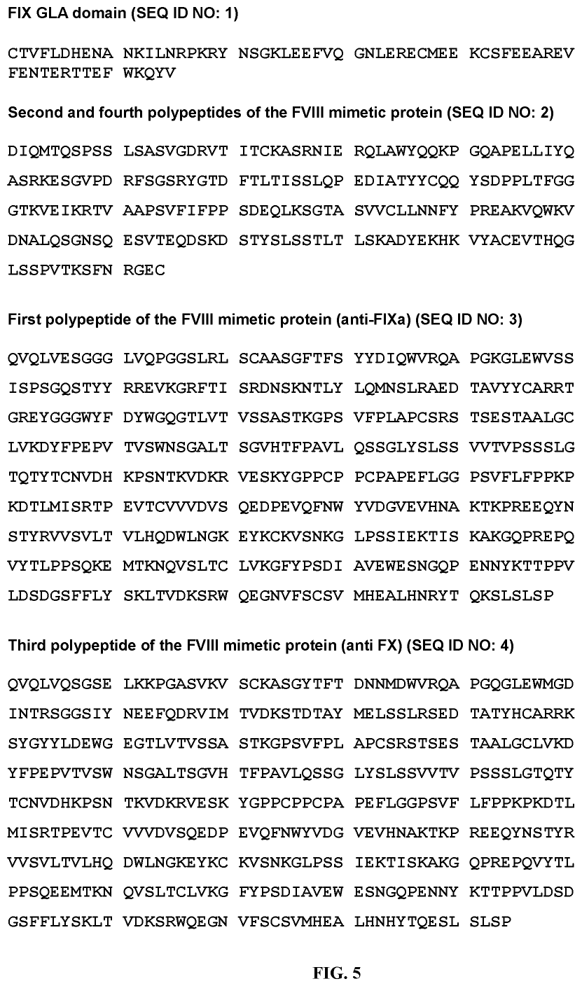

The constructed vectors are transiently co-expressed in host cells such as HEK293H cells to assemble the FVIII mimetic protein comprising the anti-human FIXa antibody H chain, the anti-human FX antibody H chain, the common shared light chain, and one or more GLA domains operably linked to the heavy or light chains. In total, 63 combinations are generated (see FIGS. 4A and 4B). As a comparative control, a bispecific antibody having the anti-human FIXa antibody H chain, the anti-human FX antibody H chain and the common shared light chain is prepared.

FVIII mimetic proteins in the cell culture supernatant are purified by a method known to those skilled in the art using protein A column.

The FXa generation-promoting activities of the FVIII mimetic proteins are by the method described below. All reactions are performed at room temperature.

Five μL of FVIII mimetic protein solution diluted with Tris-buffered saline containing 0.1% bovine serum albumin (hereafter referred to as TBSB) is mixed with 2.5 μL of 27 ng/mL Human Factor IXa beta (Enzyme Research Laboratories) and 2.5 μL of 6 IU/mL of human blood coagulation factor IX (Novact® M (Kaketsuken)), and then incubated in a 384-well plate at room temperature for 30 minutes.

The enzyme reaction in this mixed solution is initiated by adding 5 μL of 24.7 μg/mL of Human Factor X (Enzyme Research Laboratories), and ten minutes later, 5 μL of 0.5 M EDTA is added to stop the reaction. The coloring reaction is initiated by adding 5 μL of coloring substrate solution. After a 50-minute coloring reaction, the change in absorbance at 405 nm is measured using the SpectraMax 340PC384 (Molecular Devices). F.Xa generation-promoting activity is indicated as the value obtained by subtracting the absorbance of the antibody-free reaction solution from the absorbance of the antibody-supplemented reaction solution.

TBCP (TBSB containing 93.75 μM synthetic phospholipid solution (SYSMEX CO.), 7.5 mM CaCl2, and 1.5 mM MgCl2) is used as the solvent for Human Factor IXa, Novact® M, and Human Factor X. A coloring substrate solution (N-benzoyl-L-isoleucyl-L-glutamyl-glycyl-L-arginine-p-nitroaniline hydrochloride (S-2222™; (CHROMOGENIX)) is dissolved in purified water at 1.47 mg/mL, and then used in this assay.

The F.Xase inhibitory action of the FVIII mimetic proteins is measured by assaying the effects on F.X activation by F.IXa in the presence of F.VIIIa using the following method. All reactions are performed at room temperature.

Five μL of FVIII mimetic protein solution diluted with Tris-buffered saline containing 0.1% bovine serum albumin (hereafter referred to as TBSB) is mixed with 2.5 μL of 80.9 ng/mL Human Factor IXa beta (Enzyme Research Laboratories), and then incubated in a 384-well plate at room temperature for 30 minutes.

2.5 μL of 1.8 IU/mL of FVIIIa (production method descried later) is further added, and 30 seconds later, the enzyme reaction in this mixed solution is initiated by adding μL of 24.7 μg/mL of Human Factor X (Enzyme Research Laboratories). Six minutes later, μL of 0.5 M EDTA is added to stop the reaction. The coloring reaction is initiated by adding 5 μL of coloring substrate solution. After a 14-minute coloring reaction, the change in absorbance at 405 nm is measured using the SpectraMax 340PC384 (Molecular Devices). FXase inhibitory action of a FVIII mimetic protein is indicated as the value obtained by subtracting the absorbance of the FVIII mimetic protein-free reaction solution from the absorbance of the FVIII mimetic protein-supplemented reaction solution.

FVIIIa is prepared by mixing 5.4 IU/mL of Kogenate® FS (Bayer HealthCare) and 1.11 μg/mL of Human alpha Thrombin (Enzyme Research Laboratories) at a volume ratio of 1:1, incubating at room temperature for one minute, and then adding 7.5 U/mL of Hirudin (Merck KgaA) at a quantity that is half the volume of the mixture solution. The prepared solution is defined as 1.8 IU/mL of FVIIIa, and one minute after addition of Hirudin, this is used for assays.

TBCP (TBSB containing 93.75 μM phospholipid solution (SYSMEX CO.), 7.5 mM CaCl2, and 1.5 mM MgCl2) is used for the solvent for Human Factor IXa, Human Factor X, Kogenate® FS, Human alpha Thrombin, and Hirudin. A coloring substrate solution S-2222™ (CHROMOGENIX) is dissolved in purified water at 1.47 mg/mL, and then used in this assay.

1. A coagulation factor VIII (FVIII) mimetic protein comprising:
a coagulating factor IX (FIX/FIXa) binding domain, said FIX/FIXa binding domain comprising a first heavy chain variable region (V_{H1}) and a first light chain variable region (V_{L1}), wherein the V_{H1} and the V_{L1} are derived from an antibody specifically binding to FIX/FIXa;
a coagulation factor X (FX) binding domain, said FX binding domain comprising a second heavy chain variable region (V_{H}) and a second light chain variable region (V_{L2}), wherein the V_{H2} and the V_{L2} are derived from an antibody specifically binding to FX; and
a membrane binding domain.

2. The FVIII mimetic protein of claim 1, further comprising a first antibody heavy chain constant region (C1H) operably linked to the V_{H1}, and a second antibody heavy chain constant region 2 (C2H) operably linked to the V_{H2}, wherein the C1H and the C2H are capable of forming a dimer.

3. The FVIII mimetic protein of claim 2, wherein the C1H and C2H comprise a hinge region, a CH2 region, and/or a CH3 region, respectively.

4. The FVIII mimetic protein of claim 2, further comprising a first antibody light chain constant region (C1L) operably linked to the V_{L1}, and a second antibody light chain constant region (C2L) operably linked to the V_{L2}.

5. The FVIII mimetic protein of claim 1, wherein the membrane binding domain is a platelet membrane binding domain.

6. The FVIII mimetic protein of claim 1, wherein the membrane binding domain is a C1, C2 domain, a PH domain, a gamma-carboxyglutamic acid-rich (GLA) domain or membrane binding domain of a platelet membrane glycoprotein.

7. The FVIII mimetic protein of claim 1, wherein the membrane binding domain is derived from a C1, C2 domain of FV or FVIII.

8. The FVIII mimetic protein of claim 1, wherein the membrane binding domain is derived from a GLA domain of Fil, FVII, FIX, FX, protein C, protein S or protein Z.

9. The FVIII mimetic protein of claim 1, wherein the membrane binding domain is derived from an apple3 platelet binding domain of FXI.

10. The FVIII mimetic protein of claim 1, wherein the membrane binding domain is derived from AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPIb-IX-V), glycoprotein VI (GPVI), glycoprotein Ia/IIa complex (GPIa/IIIa), glycoprotein IIb/IIIa complex (GPIIb/IIIa), or GPV/IIIa (GPV/IIa).

11. The FVIII mimetic protein of claim 1, wherein the membrane binding domain binds to lipid membrane through a platelet membrane protein.

12. The FVIII mimetic protein of claim 11, wherein the platelet membrane protein is AVPRIA, CCR4, CD97, CXCR4, LPAR5/GPR92, P2RY1, P2RY12, PTAFR, PTGDR, PTGIR, XPR1, PAR1, PAR4, glycoprotein Ib-IX-V complex (GPIb-IX-V), glycoprotein VI (GPVI), glycoprotein Ia/IIa complex (GPIa/IIIa), glycoprotein IIb/IIIa complex (GPIIb/IIIa), or GPV/IIIa (GPV/IIa).

13. The FVIII mimetic protein of claim 1, wherein the membrane binding domains are operably linked to the N terminal of the V_{H1} and/or V_{H}.

14. The FVIII mimetic protein of claim 2, wherein the membrane binding domains are operably linked to the C terminal of the C1H and/or C2H.

15. FVIII mimetic protein of claim 2, wherein the membrane binding domains are operably linked to the N terminal of the V_{H1} and/or V_{H2}, and to the C terminal of the C1H and/or C2H.

16. The FVIII mimetic protein of claim 1, wherein the membrane binding domains are operably linked to the N terminal of the V_{L1} and/or V_{L2}.

17. The FVIII mimetic protein of claim 4, wherein the membrane binding domains are operably linked to the C terminal of the C1L and/or C2L.

18. The FVIII mimetic protein of claim 4, wherein the membrane binding domains are operably linked to the N terminal of the V_{L1} and/or V_{L2}, and to the C terminal of the C1L and/or C2L.

19. The FVIII mimetic protein of claim 4, wherein the membrane binding domains are operably linked to:
1) the N terminal of V_{H2};
2) the C terminal of C2H;
3) both the N terminal of V_{H2} and the C terminal of C2H;
4) the N terminal of V_{H1};
5) both the N terminals of V_{H1} and V_{H2};
6) the N terminal of V_{H1} and the C terminal of C2H;
7) both the N terminals of V_{H1} and V_{H2}, and the C terminal of C2H;
8) the C terminal of V_{H1};
9) the C terminal of C1H and the N terminal of V_{H2};
10) both the C terminals of C1H and C2H;
11) both the C terminals of C1H and C2H, and the N terminal of V_{H1};
12) both the N of V_{H1} and the C terminal of C1H;
13) both the N terminals of V_{H1} and V_{H}, and the C terminal of C1H;
14) both the C terminals of C1H and C2H, and the N terminal of V_{H1};
15) both the C terminals of C1H and C2H, and both the N terminal of V_{H1} and V_{H2};
16) both the N terminals of V_{L1} and V_{L2};
17) both the N terminals of V_{L1} and V_{L2}, and the N terminal of V_{H2};
18) both the N terminals of V_{L1} and V_{L2}, and the C terminal of C2H;
19) both the N terminals of V_{L1} and V_{L2}, the N terminal of V_{H2}, and the C terminal of C2H;
20) both the N terminals of V_{L1} and V_{L2}, and the N terminal of V_{H1};
21) both the N terminals of V_{L1} and V_{L2}, and both the N terminal of V_{H1} and V_{H2};
22) both the N terminals of V_{L1} and V_{L2}, the N terminal of V_{H1}, and the C terminal of C2H;
23) both the N terminals of V_{L1} and V_{L2}, both the N terminal of V_{H1} and V_{H2}, and the C terminal of C2H;
24) both the N terminals of V_{L1} and V_{L2}, and the C terminal of C1H;
25) both the N terminals of V_{L1} and V_{L2}, the N terminal of V_{H2}, and the C terminal of C1H;
26) both the N terminals of V_{L1} and V_{L2}, and both the C terminal of C1H and C2H;
27) both the N terminals of V_{L1} and V_{L2}, both the C terminal of C1H and C2H, and the N terminal of V_{H2};
28) both the N terminals of V_{L1} and V_{L2}, the N terminal of VH1, and the C terminal of C1H;
29) both the N terminals of V_{L1} and V_{L2}, both the N terminal of V_{H1} and V_{H2}, and the C terminal of C1H;
30) both the N terminals of V_{L1} and V_{L2}, both the C terminal of C1H and C2H, and the N terminal of V_{H1};
31) both the N terminals of V_{L1} and V_{L2}, both the C terminal of C1H and C2H, and both the N terminal of V_{H1} and V_{H2};
32) both the C terminals of C1L and C2L;
33) both the C terminals of C1L and C2L, and the N terminal of V_{H1};
34) both the C terminals of C1L and C2L, and the C terminal of C2H;
35) both the C terminals of C1L and C2L, the N terminal of V_{H1}, and the C terminal of C2H;
36) both the C terminals of C1L and C2L, and the N terminal of V_{H1};
37) both the C terminals of C1L and C2L, and both the N terminals of V_{H1} and V_{H2};
38) both the C terminals of C1L and C2L, the N terminal of V_{H1}, and the C terminal of C2H;
39) both the C terminals of C1L and C2L, both the N terminals of V_{H1} and V_{H2}, and the C terminal of C2H;
40) both the C terminals of C1L and C2L, and the C terminal of C1H;
41) both the C terminals of C1L and C2L, the C terminal of C1H, and the N terminal of V_{H2};
42) both the C terminals of C1L and C2L, and both the C terminals of C1H and C2H;
43) both the C terminals of C1L and C2L, both the C terminals of C1H and C2H, and the N terminal of V_{H2};
44) both the C terminals of C1L and C2L, the N terminal of V_{H1}, and the C terminal of C1H;
45) both the C terminals of C1L and C2L, both the N terminals of V_{H1} and V_{H}, and the C terminal of C1H;
46) both the C terminals of C1L and C2L, both the C terminals of C1H and C2H, and the N terminal of V_{H1};
47) both the C terminals of C1L and C2L, both the C terminals of C1H and C2H, and both the N terminals of V_{H1} and V_{H2};
48) both the N terminals of V_{L1} and V_{L2}, and both the C terminals of C1L and C2L;
49) both the N terminals of V_{L1} and V_{L2}, both the C terminals of C1L and C2L, and the N terminal of V_{H1};
50) both the N terminals of V_{L1} and V_{L2}, both the C terminals of C1L and C2L, and the C terminal of C2H;
51) both the N terminals of V_{L1} and V_{L2}, both the C terminals of C1L and C2L, the N terminal of V_{H2}, and the C terminal of C2H;
52) both the N terminals of V_{L1} and V_{L2}, both the C terminals of C1L and C2L, and the N terminal of V_{H1};
53) both the N terminals of V_{L1} and V_{L2}, both the C terminals of C1L and C2L, and both the N terminals of V_{H1} and V_{H2};
54) both the N terminals of V_{L1} and V_{L2}, both the C terminals of C1L and C2L, the N terminal of V_{H1}, and the C terminal of C2H;
55) both the N terminals of V_{L1} and V_{L2}, both the C terminals of C1L and C2L, both of the N terminal of V_{H1} and V_{H2}, and the C terminal of C2H;
56) both the N terminals of V_{L1} and V_{L2}, both the C terminals of C1L and C2L, and the C terminal of C1H;
57) both the N terminals of V_{L1} and V_{L2}, both the C terminals of C1L and C2L, the C terminal of C1H, and the N terminal of V_{H2};
58) both the N terminals of V_{L1} and V_{L2}, both the C terminals of C1L and C2L, and both the C terminal of C1H and C2H;
59) both the N terminals of V_{L1} and V_{L2}, both the C terminals of C1L and C2L, both the C terminal of C1H and C2H, and the N terminal of V_{H2};
60) both the N terminals of V_{L1} and V_{L2}, both the C terminals of C1L and C2L, the N terminal of V_{H1}, and the C terminal of C1H;
61) both the N terminals of V_{L1} and V_{L2}, both the C terminals of C1L and C2L, both the N terminal of V_{H1} and V_{H2}, and the C terminal of C1H;
62) both the N terminals of V_{L1} and V_{L2}, both the C terminals of C1L and C2L, both the C terminal of C1H and C2H; or
63) both the N terminals of V_{L1} and V_{L2}, both the C terminals of C1L and C2L, both the N terminals of V_{H1} and V_{H2}, and both the C terminal of C1H and C2H.

20. A nucleic acid encoding the FVIII mimetic protein of claim 1.

21. (canceled)

22. (canceled)

23. (canceled)

24. A pharmaceutical composition comprising the FVIII mimetic protein of claim 1 and a pharmaceutically acceptable carrier.

25. (canceled)

26. A method for treating or reducing the incidence of bleeding, a disease accompanying bleeding, or a disease caused by bleeding in a subject, the method comprising introducing to a cell of the subject a vector comprising a nucleic acid encoding a coagulation factor VIII (FVIII) mimetic protein, said FVIII mimetic protein comprising:
a coagulating factor IX (FIX/FIXa) binding domain comprising a first heavy chain variable region (V_{H1}) and a first light chain variable region (V_{L1}), wherein the V_{H1} and the V_{L1} are derived from an antibody specifically binding to FIX/FIXa; and
a coagulation factor X (FX) binding domain, said FX binding domain comprising a second heavy chain variable region (V_{H2}) and a second light chain variable region (V_{L2}), wherein the V_{H2} and the V_{L2} are derived from an antibody specifically binding to FX; and
a membrane binding domain.

27. The method of claim 26, wherein the bleeding, a disease accompanying bleeding, or a disease caused by bleeding is hemophilia A, acquired hemophilia or von Willebrand disease.

28. The method of claim 26, wherein the cell is endothelial cell, liver cell, platelet, PBMC or hematopoietic stem cell.

29. The method of claim 26, wherein the subject is a human.

30. The method of claim 26, wherein the vector is an episomal expression vector.

31. The method of claim 26, wherein the vector is a donor vector for gene knockin.

32. The method of claim 26, wherein the vector is introduced to the cell via a virus.

33. The method of claim 32, wherein the virus is an adeno-associated virus, a retrovirus or a lentivirus.

34. The method of claim 26, further comprising introducing to the cell a site-specific nuclease.

35. The method of claim 34, wherein the site-specific nuclease is selected from a CRISPR nuclease, a TALEN, a DNA-guided nuclease and a Zinc Finger nuclease.

36-43. (canceled)
